(19) United States
(12) Patent Application Publication
(10) Pub. No.: US 2009/0093509 A1
(43) Pub. Date: Apr. 9, 2009

(54) Methods and Compositions for the Treatment of Pruritus

(76) Inventors: Tahir Nazir, Cambridge (GB); Andrew Douglas Baxter, Cambridge (GB); Alan Rothaul, Cambridge (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/247,618

(22) Filed: Oct. 8, 2008

(30) Foreign Application Priority Data
Oct. 8, 2007 (GB) 0719613.2
May 22, 2008 (GB) 0809319.7

(51) Int. Cl. A61K 31/485

(52) U.S. Cl. 514/282

(57) ABSTRACT

The present invention is based on the discovery that a nalmefene salt or a peripherally acting analogue of nalmefene is capable of providing long-acting activity against pruritus, when applied topically. In addition, treatment with a nalmefene salt or peripherally acting analogue of nalmefene, can provide disease-modifying effects against the chronic symptoms of atopic dermatitis, including reductions of inflammatory cell infiltrate into the skin, epidermal hyperplasia and trans-epidermal water loss. Compositions of the invention have an inhibitory effect on the itch-scratch-cycle, leading to both relief of pruritus and disease-modifying activity.

### FIELD OF THE INVENTION

This invention relates to compositions for the treatment of pruritus.

### BACKGROUND OF THE INVENTION

Pruritus is an itch or a sensation that makes a person want to scratch. Pruritus can cause discomfort and be frustrating. If it is severe, it can lead to sleeplessness, anxiety and depression. Pruritus can be a part of skin diseases, or internal disorders, or due to faulty processing of the itch sensation within the nervous system.

There are many skin diseases, such as urticaria (hives), varicella (chicken pox), and eczema which may have itching associated with a rash. Some skin conditions only have symptoms of pruritus without having an obvious rash. Dry skin can itch, especially in the winter, with no visible signs of a rash. Some parasitic infestations such as scabies, worms, and lice may be very itchy. Itchy, pigmented moles may be a sign of a malignant change. Pruritus can also be drug-related and age-related (senile pruritus).

Pruritus may be a manifestation of an internal condition. The most common example are kidney failure, and cholestatic, and uremic diseases. Some types of liver disease, such as hepatitis, thyroid disease including both hyper- and hypo-thyroid hormone levels, some blood disorders such as lymphomas, iron-deficiency anemia, polycythemia vera, multiple myeloma, and neurologic conditions such as pinched nerves and post herpetic neuralgia, can cause itch. Infectious diseases, such as HIV, viral infections, such as chicken pox, and bacterial or fungal infections can cause severe itching. Other pruritus-causing conditions include cutaneous vasculitis, primary localized cutaneous amyloidosis (PLCA), prurigo nodularis, and lichen striatus.

One of the conditions in which pruritus is of particular importance is in atopic dermatitis (AD). The population of patients with AD has been estimated as in the order of 40 million. Pruritus is an implicit feature of AD, with all patients experiencing some level of “itch.” The pruritic component of AD is recognised as a very significant and currently untreated component of the condition. Surveys suggest that many patients regard the severity of itch rather than the appearance of their skin lesions as the primary measure of disease severity. It is broadly accepted that a phenomenon called the “ditch-scratch” cycle is a key component of AD. Itching results in scratching, causing greater inflammation and breakdown of the skin barrier, with greater opportunity for dermal infection. This in itself results in greater itching. Thus, the “itch-scratch” syndrome is regarded as a vicious cycle implicated in the worsening of AD. Night-time itch is experienced by many AD sufferers, resulting in the loss of restful sleep and overall reduction in quality of life; this is particularly significant in children.

It has long been known that systemic opioids cause itch as a side-effect. For some time, this was regarded uniquely as a result of activation of central nervous system opioid receptors. It has also been established in a number of clinical studies that systemically administered opioid antagonists (e.g., naloxone and naltrexone) have anti-pruritic activity in a range of conditions including AD. These clinical studies have also indicated that, whilst effective against pruritus, systemic administration of opioid antagonists has been associated with side-effects characterised as opioid withdrawal-like (e.g., nausea, agitation and dysphoria).

Nalmefene (6-methylene-6-desoxy-N-cyclopropylmethyl-14-hydroxydihydronormorphine) is a long-acting, orally available, potent opioid antagonist with pure and selective antagonist activity. Nalmefene hydrochloride (Revex®) is available as a sterile solution for intravenous, intramuscular and subcutaneous administration, indicated for the reversal of opioid drug effects, including respiratory depression.

The use of systemic nalmefene has been proposed for the treatment of a variety of diseases, including autoimmune diseases, such as multiple sclerosis, arthritic and inflammatory diseases, and mental, emotional or stress disorders. In addition, nalmefene has been disclosed to treat mast cell-mediated disorders, such as allergic rhinitis, and also dermatological diseases. U.S. Pat. No. 4,946,848 describes treating pruritus, whether antigen-induced or disease-induced, by orally administering 1 to 25 mg nalmefene.

U.S. Pat. No. 4,923,875 discloses the utility of topical nalmefene in treating urticaria, various eczemas, and other mast cell-mediated dermatological disorders. This specification includes a report of a study in which pruritus was treated by application of a topical gel comprising nalmefene free base. It is stated that “Preferred inert vehicles or carriers . . . promote rapid release and absorption of the drug upon application to the affected skin areas”. The specification refers to nalmefene and its salts and esters, but all the Examples utilise the free base.

### SUMMARY OF THE INVENTION

The present invention is based on the discovery that a nalmefene salt or a peripherally acting analogue of nalmefene is capable of providing long-acting activity against pruritus, as evidenced by testing in a recognised animal model of AD (human apolipoprotein C1 transgenic mice), when applied topically. These effects clearly demonstrate a long duration of action (>8 hours), indicating that nalmefene when delivered topically may be suitable for the once or twice daily treatment of itch. In addition, and surprisingly, treatment with a nalmefene salt or peripherally acting analogue of nalmefene, can provide disease-modifying effects against the chronic symptoms of AD studied in this model, including reductions of inflammatory cell infiltrate into the skin, epidermal hyperplasia and trans-epidermal water loss. This indicates that compositions of the invention have an inhibitory effect on the itch-scratch-cycle, leading to both relief of pruritus and disease-modifying activity.

In addition, compositions of the invention can be used to treat acute itchy flares, chronic itch and night-time itch, e.g., associated with both acute and chronic AD. They can also be used for the prevention of AD and pruritus flares. Based on their efficacy and pharmacodynamics, nalmefene salts can be used at a lower dose than other opioid antagonists (such as naloxone and naltrexone). They may also be used in combination with other commonly used treatments of pruritus, including corticosteroids, calcineurin inhibitors and anti-histamines, or with a local anaesthetic, a vasoconstrictor, an anti-depressant, an antifungal or an antibiotic.

Opioid antagonists, even when applied topically, provide efficacy but are susceptible to causing unwanted CNS side-effects that would limit their use in AD, particularly in children. These side-effects can be managed by the topical application of a nalmefene salt or a peripherally acting analogue of nalmefene when controlled delivery of the compound allows retention in/on the skin and minimizes systemic and ultimately central exposure. This is demonstrated by Franz Cell experiments where certain topical formulations of nalmefene are shown to pass slowly through synthetic and skin membranes.

According to the present invention, pruritus is treated by the use of a composition comprising a compound of general formula (1):

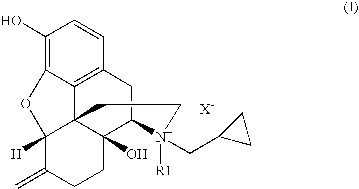

wherein R1 is H, such as in a pharmaceutically active salt of nalmefene itself, or C₁-C₄ alkyl or C₁-C₄ alkylcycloalkyl in the peripherally acting analogues; and X is a counterion.

One aspect of the invention is the treatment of night-time pruritus. Another is the composition as such, independent of use, when comprising water or an aqueous phase and having an acidic pH. Yet another is the use of the compounds of formula (1) when R1 is not H and the condition to be treated is pruritus.

In another aspect, the invention features a controlled release formulation that includes a compound of formula (1) formulated for topical administration (e.g., as an ointment, lotion, or cream).

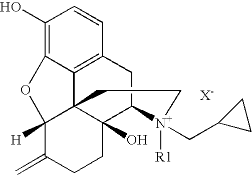

In formula (1), R1 is H, C₁-C₄ alkyl or C₁-C₄ alkylcycloalkyl; and X is a counterion of any pharmaceutically acceptable acid (e.g., iodide or chloride). In one embodiment, the compound of formula (1) is a nalmefene salt. The compound of formula (1) can be formulated, for example, at a concentration between 0.1% and 5% w/w (e.g., between 0.5% and 2% w/w, or 1% w/w) and can optionally include an emollient or an antiseptic agent. Furthermore, the composition of formula (1) can be formulated for controlled release (e.g., formulated to release effective amounts of a compound of formula (1) onto or into the dermis over a period of 2, 4, 6, 8, 12, or more hours).

The controlled release formulation of the invention can contain any of the following properties, alone, or in combination:
it can be formulated such that upon topical administration to a human at least 10% of the drug is retained within the skin for at least 4, 6, 8, or 12 hours, or more after administration;
it can be formulated such that upon topical administration to a human the systemic exposure of the human to the nalmefene salt is less than 20 ng/ml (e.g., less than 10 ng/ml or 5 ng/ml);
it can be formulated with a penetration modifier (e.g., isopropyl myristate);
it can be formulated such that relief from pruritus from the treatment occurs less than 2 hours (e.g., less than 1 hour) after administration and is sustained for at least 4 hours (e.g., for at least 6, 8, or 12 hours, or more) after administration; and
it can be formulated at a pH of between 3 and 7 (e.g. a pH of 3, 3.5, 4, 4.5, 5, 5.5, 6, 6.5, or 7);

The composition of formula (1) can also be formulated with an additional active agent, such as those described herein.

The invention also features a method of treating pruritus in a subject (e.g., a human adult or child) by administering any of the above formulations of a compound of formula (1). The subject can be between 0 and 5, 5 and 13, 13 and 18 years old, or older. The administration can occur, for example, substantially immediately prior to sleep. The pruritus being treated can be associated, for example, with atopic dermatitis, psoriasis, urticaria, varicella, eczema, contact dermatitis, dry skin, parasitic infestation, an internal condition (e.g., hepatitis, thyroid disease, a blood disorder, a neurological condition, and a malignant or infectious disease) a viral, fungal, or bacterial infection, or a drug-related or age-related condition. If desired, the formulations of the invention can also be administered in combination (e.g., simultaneous with, or within 1, 2, 6, 8, or 12 hours of) an additional active agent, such as a corticosteroid (e.g., hydrocortisone, betamethasone, flumethasone or prednisolone), a local anesthetic (e.g., lidocaine), a calcineurin antagonist (e.g., pimecrolimus or tacrolimus), a polydocanol, a terpene (e.g., camphor or menthol), an anti-histamine (e.g., trimeprazine), or an antibiotic.

By “pruritus” is meant a condition which causes an itch or sensation that induces a subject to desire to scratch. Such conditions include, for example, dermal inflammatory disorders, dry skin, and conditions caused by parasitic infections, microbial infections (e.g., bacterial, viral, or fungal infections), internal disorders (e.g., hepatitis, thyroid disease, a blood disorder, a neurological condition or a malignant or infectious disease), age, or by the ingestion of pharmaceutical compounds.

By “dermal inflammatory disorders” are meant immunoinflammatory conditions affecting skin. “Dermal inflammatory disorders” include, for example, atopic dermatitis, hand dermatitis, and actinic keratosis, psoriasis, acute febrile neutrophilic dermatosis, eczema (e.g., asteatotic eczema, dyshidrotic eczema, vesicular palmoplantar eczema), balanitis circumscripta plasmacellularis, balanoposthitis, Behcet's disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

By “treating” is meant administering or prescribing a composition for the treatment or prevention of pruritus.

By “subject” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

By “effective amount” is meant the amount of a compound, required to treat or prevent pruritus in a clinically relevant manner. An effective amount of the active compounds used to practice the present invention for therapeutic treatment of conditions caused by or contributing to pruritus varies depending upon the manner of administration, the age, body weight, and general health of the subject. Ultimately, the prescribers will decide the appropriate amount and dosage regimen. The term effective amount is also meant to include an amount sufficient to provide sufficient relief to allow the subject to sleep for at least 6 hours undisturbed by pruritus.

By “more effective” is meant that a method, composition, or kit exhibits greater efficacy, is less toxic, safer, longer-acting, more convenient, better tolerated, less expensive, or provides more treatment satisfaction than another method, composition, or kit with which it is being compared. Efficacy may be measured by a skilled practitioner using any standard method that is appropriate for a given indication.

By “controlled release” is meant that the therapeutically active component is released from the formulation at a controlled rate such that therapeutically levels (but below toxic levels) of the component are maintained on or in the dermis over an extended period of time ranging from e.g., about 4 hrs to about 24 hours or more, thus, providing, for example, 4, 5, 6, 7, 8, 9, 10, 11, 12 hour or a 24 hour or more dosage form.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein.

### DETAILED DESCRIPTION OF THE INVENTION

In general, the invention features methods, compositions, and kits for providing prolonged relief from pruritus associated with dermal inflammatory disorders and other conditions. The invention features controlled release formulations of the compound of formula (1), e.g., a nalmefene salt. Controlled release is obtained, for example, due to the salt form of formula (1), through the use of certain penetration modifiers (e.g., isopropyl myristate), and through maintaining an acidic pH in the formulation.

### Pruritus

The invention features compositions, kits, and methods for treating pruritus, a condition that causes an itch or sensation that induces a subject to desire scratching. Such conditions include, for example, dermal inflammatory disorders, dry skin, and conditions caused by parasitic infections, microbial infections (e.g., bacterial, viral, or fungal infections), internal disorders (e.g., hepatitis, thyroid disease, a blood disorder, a neurological condition or a malignant or infectious disease), age, or by the ingestion of pharmaceutical compounds. Dermal inflammatory disorders are characterized by the inappropriate activation of the body's immune defenses at or near the surface of the skin. Examples of dermal inflammatory disorders are psoriasis, atopic dermatitis, hand dermatitis, and actinic keratosis, each of which is described in more detail below.

The invention features the long-term treatment and prevention of pruritus in order to allow, for example, the prolonged duration of relief necessary for a night-long comfortable sleep.

### Atopic Dermatitis

The methods, compositions, and kits of the invention may be used for the treatment of atopic dermatitis. If desired, one or more atopic dermatitis agents typically used to treat atopic dermatitis may also be used in the methods, composition, and kits of the invention. Such agents include topical and systemic non-steroidal immunophilin-dependent immunosuppressants (e.g., cyclosporine, tacrolimus, pimecrolimus, ABT-281, and ISAtx247), topical glucocorticosteroids (e.g., clobetasol, triamcinolone, betamethasone, hydrocortisone, halobetasol, diflorasone, mometasone, halcinonide, and fluticasone), systemic glucocorticosteroids (e.g., prednisone and dexamethasone) and antihistamines (e.g., hydroxyzine, loratadine, cetirizine, diphenhydramine, cyproheptadine, and fexofenadine).

### Psoriasis

The methods, compositions, and kits of the invention may be used for the treatment of psoriasis. If desired, one or more antipsoriatic agents typically used to treat psoriasis may also be used in the methods, compositions, and kits of the invention. Such agents include biologics (e.g., alefacept, infliximab, adalimumab, efalizumab, etanercept, and CDP-870), small molecule immunomodulators (e.g., VX 702, SCIO 469, doramapimod, RO 30201195, SCIO 323, DPC 333, pralnacasan, mycophenolate, and merimepodib), non-steroidal immunophilin-dependent immunosuppressants (e.g., cyclosporine, tacrolimus, pimecrolimus, ABT-281, and ISAtx247), vitamin D analogs (e.g., calcpotriene, calcipotriol), psoralens (e.g., methoxsalen), retinoids (e.g., acitretin, tazarotene), DMARDs (e.g., methotrexate), anthralin, topical glucocorticosteroids (e.g., clobetasol, triamcinolone, betamethasone, hydrocortisone, halobetasol, diflorasone, mometasone, halcinonide, fluticasone), and systemic glucocorticosteroids (e.g., prednisone, dexamethasone).

### Hand Dermatitis

The methods, compositions, and kits of the invention may be used for the treatment of hand dermatitis. If desired, one or more hand dermatitis agents typically used to treat hand dermatitis may also be used in the methods, composition, and kits of the invention. Such agents include topical and systemic non-steroidal immunophilin-dependent immunosuppressants (e.g., cyclosporine, tacrolimus, pimecrolimus, ABT-281, and ISAtx247), topical glucocorticosteroids (e.g., clobetasol, triamcinolone, betamethasone, hydrocortisone, halobetasol, diflorasone, mometasone, halcinonide, and fluticasone), systemic glucocorticosteroids (e.g., prednisone and dexamethasone) antihistamines (e.g., hydroxyzine, loratadine, cetirizine, diphenhydramine, cyproheptadine, and fexofenadine), and emollients, ointments, humectants, and lotions.

### Actinic Keratosis

The methods, compositions, and kits of the invention may be used for the treatment of actinic keratosis. If desired, one or more agents typically used to treat dermal inflammatory disorders may be used in the methods, composition, and kits of the invention. Such agents include chemotherapeutic agents (e.g., 5-fluorouracil, imiquimod), non-steroid inflammatory agents (e.g., diclofenac), topical retinoids (e.g., adapalene), and photodynamic therapy using topical aminolevulinic acid.

### Compounds

Compounds of formula (1) are salts. X is preferably a halide but can represent the counterion of any pharmaceutically acceptable acid, including inorganic acids, e.g., a sulphate, methosulphate or phosphate, and organic acids, such as a citrate, succinate, tartarate, fumarate, maleate, acetate, methanesulphonate or the like. Preferably, the salt is of nalmefene itself (R1 is H), e.g., nalmefene hydrochloride.

Nalmefene (6-methylene-6-desoxy-N-cyclopropylmetlhyl-14-hydroxydihydronormorphine) is a long-acting, orally available, potent opioid antagonist with pure and selective antagonist activity. Nalmefene hydrochloride (Revex®) is available as a sterile solution for intravenous, intramuscular and subcutaneous administration, indicated for the reversal of opioid drug effects, including respiratory depression.

Nalmefene has many chiral centres. Any reference herein to nalmefene or peripherally acting analogues should be understood as a reference to any enantiomer or mixture thereof. Any enantiomer may be substantially free of others, e.g., in an enantiomeric excess of at least 80%, preferably at least 90% and more preferably at least 95%. Similarly, any mixture of diastereomers may be substantially free of the other. The form drawn above as (1) is preferred. Nalmefene may be in the form of any pharmaceutically acceptable salt, as described above. Such forms are known to those of ordinary skill in the art.

### Administration

It may be desirable to administer to the subject other compounds, such as a corticosteroid, humectants, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Therapies of the invention are also useful for the treatment of pruritis in combination with other agents—either biologics or small molecules—that modulate the immune response to affect disease. Such agents include those that deplete key inflammatory cells, influence cell adhesion, or influence cytokines involved in immune response. This last category includes both agents that mimic or increase the action of anti-inflammatory cytokines such as IL-10, as well as agents inhibit the activity of pro-inflammatory cytokines such as IL-6, IL-1, IL-2, IL-12, IL-15 or TNF\tilde{□}. Agents that inhibit TNF□ include etanercept, adelimumab, infliximab, and CDP-870. Small molecule immunodulators include, e.g., p38 MAP kinase inhibitors such as VX 702, SCIO 469, doramapimod, RO 30201195, SCIO 323, TACE inhibitors such as DPC 333, ICE inhibitors such as pranalcasan, and IMPDH inhibitors such as mycophenolate and merimepodib.

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the subject, the stage and type of the subject's disease, and how the subject responds to the treatment. Additionally, a person having a greater risk of developing an inflammatory disease (e.g., a person who is undergoing age-related hormonal changes) may receive treatment to inhibit or delay the onset of symptoms.

The compounds of the invention may be topically administered. The amount of active ingredient that is used can be chosen by the skilled person having regard to the usual factors.

For use, the active agent is typically formulated, e.g., with one or more of conventional diluents or carriers, as a dressing, or as a medicament adapted to be delivered topically. Such formulations are preferably designed to retard the passage of the compound into the systemic circulation and are chosen according to such considerations as the potency of the drug, the severity of the condition and the route of administration. For example, the compounds of the invention can be formulated to limit systemic exposure to less than, for example, 20 ng/ml, less than 10 ng/ml or 5 ng ml or less. Systemic exposure can be measured using standard bioanalytical methods.

Topical formulations that can be used with the compounds of the invention include, without limitation, creams, foams, lotions, gels, and ointments. In particular, the invention features controlled release formulations of the compounds of the invention such that relief from pruritus is sustained for long periods of time (e.g., overnight). Sustained release is achieved in part, or in whole, by the use of a salt form of the compounds of the invention (e.g., the compound of formula (1)). Sustained release can be further achieved, for example, by providing a pH of between 3 and 7 (e.g., between 4 and 7, between 3 and 6, and between 4 and 6) and by the addition of a penetration modifier to the topical formulation (e.g., isopropyl myristate). The weight ratio of a penetration enhancer such as isopropyl myristate and water is, for example, between 30:1 and 5:1

The formulations can include various conventional colorants, fragrances, thickeners (e.g., xanthan gum) preservatives, emollients (e.g., hydrocarbon oils, waxes, or silicones), demulcents, solubilizing excipients, dispersants, penetration enhancers, plasticizing agents, preservatives, stabilizers, demulsifiers, wetting agents, emulsifiers, moisturizers, astringents, deodorants, and the like can be added to provide additional benefits and improve the feel and/or appearance of the topical preparation.

For example, a composition of the invention may comprise conventional components, e.g., as listed in the Example under “Property”. Preferred components for use in the invention include penetration modifiers, e.g., isopropyl myristate, isopropyl palmitate, isopropyl hexanoate; humectants (and moisturizers), e.g., glycerol, propylene glycol, urea, sorbitol, mannitol, xylitol and other sugar derivatives, lactic acid and salts, hyaluronates and glycosaminoglycans, dextrans, amino acids, pyrrolidone-5-carboxylic Acid (PCA) and salts and other natural moisturizing factor in, glutamic acid; preservatives, e.g., phenoxyethanol, benzyl alcohol, parahydroxybenzoates (with or without EDTA), phenols and derivatives and sorbic acid and salts; and thickening agents e.g., natural gums (such as xanthan gum, carrageenan), cellulose derivatives (such as HEC, SCMC, MC, HPMC, carbomers).

The pH of the formulations of the invention are greater than 3 and less than 7. For example, the pH can be 3-7, 3-6, 4-7, 4-6, or 4-5. The pH of the solution and the measured pH of the topical composition may not be the same. For the purposes of this specification, either pH may be relevant but it is preferred that the pH is defined as that in the aqueous phase and not in the final cream. The pH of a 0.1% w/v aqueous solution of nalmefene hydrochloride is 6.13 at 22.5° C.: the corresponding values for 1% and 5% solutions are 5.76 and 4.66.

The acidity of the composition ensures that nalmefene remains in the salt form and delays release of drug into the skin and retards transdermal delivery. It will be appreciated that the salt used in this invention may be provided as an aqueous solution which is then mixed with other ingredients to form a topical composition. For example, due in part or in whole to the acidic pH the formulations of the invention can result in 5%, 10%, 15%, 20%, 25%, 30%, 40%, or 50%, or greater retention of the drug within the skin for at least 4 hours (e.g., 5, 6, 7, 8, 9, 10, or 12 hours, or longer). Skin retention can be measured, for example by in vitro Franz cell skin stripping experiments.

Desirably, the compound of formula (1) is formulated such that relief from pruritus from the treatment occurs less than 2 hours after administration and is sustained for at least 4 hours (e.g., for at least 6, 8, 12 hours, or more) after administration.; and

Topical delivery can introduce significant concentrations of the compound of formula (1) into/onto the skin whilst reducing CNS and peripheral side-effects. In a composition of the invention, the salt is typically present at a concentration between 0.1% and 5%, preferably between 0.5% and 2% w/w. The % of ionised drug in the composition is more than 90%, preferably more than 95%, most preferably more than 99%.

It will often be advantageous to use a compound of formula (1) in combination with another drug used for pruritus or atopic dermatitis therapy. Such another drug may be a local anaesthetic, corticosteroid, calcineurin antagonist or anti-infective. Other suitable combinations may include anti-histamines, including either or both sedating or non-sedating agents, antidepressants, antibacterials, antifungals. Drugs when used in combination with compounds of formula (1) can be administered either topically or systemically, in the same or a different formulation.

A composition of the invention preferably has control-release characteristics. Such characteristics can be determined by an objective study such as an in vitro Franz cell experiment.

### Dosages

The dosage of each compound of the claimed compounds depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular subject may affect dosage used. For example, a daily dose of the active agent may be 1 mg to 2 g (e.g., 1 mg to 1 g or 1 mg to 0.5 g)

Continuous daily dosing with the compounds of the invention may not be required. A therapeutic regimen may require cycles, during which time a drug is not administered, or therapy may be provided on an as needed basis during periods of acute inflammation. Further, the formulations of the invention can be administered prior to periods of expected prolonged need (e.g., prior to sleep). The dosage of active agent may be 0.5 mg to 4 mg/cm³ (e.g. 1 mg to 2 mg/cm³).

A composition of the invention provides controlled release into the stratum corneum, to form a drug depot from which the drug is released slowly into the epidermis. These release properties result in the unexpectedly long duration of action. Further advantages include favourable PK/PD and low systemic exposure. Preferably the systemic exposure of a subject to nalmefene is less than 20 ng/ml, more preferably less than 10 ng/ml or less than 5 ng/ml.

The properties of a composition of the invention are such that relief from pruritis can be provided for a period of at least 4 hours after administration. This period can in fact be at least 6, 8, or 12 hours, or more. As a consequence of the long lasting anti-pruritic effect, a composition of the invention may be administered once or twice daily.

For the treatment of night-time itch, a composition of the invention is preferably administered to the skin immediately before sleep. The nature of the composition and/or the active agent are such that the latter's anti-pruritic activity can last through at least a substantial part of what would normally be considered a good night's sleep, e.g., at least 4, 6, or 8 hours, or more. Thus, the need for medication during the night can be avoided. The facts that night-time pruritis is a particular problem in children, and children generally need more sleep than adults, make the invention particularly suitable for paediatric use.

### Kits

In general, kits of the invention contain a compound of formula (1) (e.g., nalmefene HCl). The kits of the invention can also contain instructions for the administration of the compound of formula (1). Kits of the invention can also contain instructions for administering an additional pharmacologically acceptable substance. Such an additional substance can also be optionally included in the same kit, and for example, be the same formulation as a compound of formula (1).

The following examples illustrate the invention.

### EXAMPLES

The following components were formulated:

Cream 1

| Item | Property | % w/w |
|---|---|---|
| Nalmefene hydrochloride | Active | 1.108 |
| Emulsifying Ointment BP | Cream Base | 30.0 |
| Glycerol | Humectant | 5.0 |
| Phenoxyethanol | Preservative | 6.4 |
| Isopropyl Myristate | Penetration modifier | 5.0 |
| Purified Water BP | Diluent | 52.492 |

Cream 2

| Item | Property | % w/w |
|---|---|---|
| Nalmefene hydrochloride | Active | 1.108 |
| Emulsifying Ointment BP | Cream Base | 30.0 |
| Glycerol | Humectant | 5.0 |
| Phenoxyethanol | Preservative | 1.0 |
| Isopropyl Myristate | Penetration modifier | 5.0 |
| Natrosol 250HX | Thickener | 0.2 |
| Purified Water BP | Diluent | 57.692 |

Cream 3

| Item | Property | % w/w |
|---|---|---|
| Nalmefene hydrochloride | Active | 1.108 |
| Emulsifying Ointment BP | Cream Base | 30.0 |
| Glycerol | Humectant | 5.0 |
| Phenoxyethanol | Preservative | 1.0 |
| Natrosol 250HX | Thickener | 0.2 |
| Purified Water BP | Diluent | 62.692 |

Cream 4

| Item | Property | % w/w |
|---|---|---|
| Nalmefene hydrochloride | Active | 1.108 |
| Emulsifying Ointment BP | Cream Base | 30.0 |
| Glycerol | Humectant | 5.0 |
| Phenoxyethanol | Preservative | 6.4 |
| Purified Water BP | Diluent | 57.492 |

Ointment

| Item | Property | % w/w |
|---|---|---|
| Nalmefene hydrochloride | Active | 1.0 |
| Emulsifying Ointment BP | Cream Base | 99.0 |

These formulations were tested in in vitro Franz cell experiments and an in vivo mouse model of pruritus, and demonstrated a high and controlled-released dermal load, with limited systemic exposure. The details of these experiments are shown below.

### Franz Cell Study

In vitro percutaneous absorption of nalmefene hydrochloride from two cream formulations through human dermatomed skin (400 μm, n=6) was assessed over a 24 hour exposure period. Cream formulation was dosed at 10 mg/cm² skin in a static Franz cell with a 0.5 ml receiver fluid volume. The distribution of nalmefene within the test system following the 24 hour exposure was also determined. Samples were analysed by HPLC-UV.

| | % absorbed | | |
|---|---|---|---|
| Sample | Epidermis | Dermis | Receiver fluid |
| Cream 1 | 7.87 ± 1.74 | 4.46 ± 1.39 | 3.12 ± 0.89 |
| Cream 4 | 8.91 ± 1.37 | 2.70 ± 0.77 | 2.31 ± 0.81 |

These data indicate that around 12% of the applied dose is retained in the skin after 24 hours, whereas a relatively small proportion passes through the skin.

In vivo Mouse Model of Pruritus Associated With Atopic Dermatitis

Human Apolipoprotein C1 transgenic mice exhibit clinical signs of atopic dermatitis at 6-8 weeks of age. These signs include skin scaling, lichenification and excoriations, and are also associated with scratching behaviour (pruritus). The severity and duration of scratching behaviour are related to the severity of the atopic dermatitis lesion score.

Groups of up to 8 male or female ApoC1 mice were treated with a single, 0.2 ml topical dose of Cream 1. Baseline scratching behaviour was established for 12 hours prior to treatment, and then monitored for 12 hours immediately following treatment. Results were compared with those for groups of untreated or placebo cream-treated control animals, and show that topical treatment with Cream 1 has a rapid and long-lasting effect on pruritus. The novel composition has a significant anti-pruritic effect for up to 12 hours, in this model.

We claim:

1. A method of treating pruritus in a subject by topically administering to said subject a composition comprising a compound of formula (1), 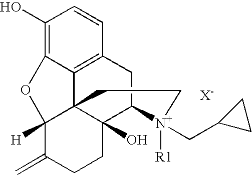
wherein R1 is H, C₁-C₄ alkyl or C₁-C₄ alkylcycloalkyl;
X is a counterion of any pharmaceutically acceptable acid; and
wherein said compound of formula (1) is present in said composition at a concentration of between 0.1% and 5% w/w, and the relief from pruritus from said treatment is sustained for at least 4 hours after administration.

2. The method of claim 1, wherein said relief from pruritus from said treatment is sustained for at least 6 hours.

3. The method of claim 1, wherein at least 10% of said compound of formula (1) is retained within the skin 4 hours after administration.

4. The method of claim 1, wherein the systemic exposure of said subject to said compound of formula (1) is less than 20 ng/ml of nalmefene.

5. The method of claim 1, where said composition comprises between 0.5 to 2% w/w of said compound of formula (1).

6. The method of claim 1, wherein said subject is between 0 and 18 years old.

7. The method of claim 1, wherein the pH of said composition is between 3 and 7.

8. The method of claim 7, wherein said pH is between 4 and 6.

9. The method of claim 1, wherein said compound of formula (1) is selected from nalmefene methiodide and nalmefene hydrochloride.

10. The method of claim 1, wherein said composition further comprises isopropyl myristate.

11. The method of claim 1, wherein said composition further comprises an emollient or an antiseptic agent.

12. The method of claim 1, wherein said composition further comprising a second active agent.

13. The method of claim 12, wherein said active second agent is selected from the group consisting of a corticosteroid, a local anesthetic, a calcineurin antagonist, polydocanol, a terpene, an anti-histamine, and an antibiotic.

14. The method of claim 1, wherein said pruritus is associated with a condition selected from the group consisting of atopic dermatitis, psoriasis, urticaria, varicella, eczema, contact dermatitis, dry skin, a parasitic infestation, hepatitis, thyroid disease, a blood disorder, a neurological condition, a malignant disease, an infectious disease, a viral infection, a fungal infection, a bacterial infection, a drug-related condition and an age related condition.

15. The method of claim 1 wherein the relief from pruritis from said treatment occurs less than 2 hours after administration.

16. The method of claim 1 wherein less than 5% of the delivered dose of said compound of formula (1) has passed through the skin 4 hours after said treatment.

17. A composition comprising a compound of formula (1) formulated for administration to a human as an ointment, lotion, or cream, wherein said compound of formula (1) is formulated such that upon topical administration to a human: a) at least 10% of said compound of formula (1) is retained within the skin 4 hours after administration, and/or b) the systemic exposure of said human to said compound of formula (1) is less than 100 μg/ml;
and wherein said compound of formula (1) is formulated in said composition at a concentration of between 0.1% and 5% w/w.

18. The composition claim 17, wherein said compound of formula (1) is formulated such that upon topical administration to a human the systemic exposure of said human to said compound of formula (1) is less than 20 ng/ml.

19. The composition of claim 17, wherein said compound of formula (1) is formulated at a pH of between 3 and 7.

20. The composition of claim 17, wherein said compound of formula (1) is nalmefene methiodide or nalmefene hydrochloride.

21. The composition of claim 17, wherein said composition comprises isopropyl myristate.

22. The composition of claim 17, wherein said composition comprises an emollient or an antiseptic agent.

23. The composition of claim 17, further comprising a second active agent.

24. The composition of claim 23, wherein said second agent is selected from the group consisting of a corticosteroid, a local anesthetic, a calcineurin antagonist, polydocanol, a terpene, an anti-histamine, and an antibiotic.

25. A composition comprising a compound of formula (1) formulated for administration to a human as an ointment, lotion, or cream, wherein said compound of formula (1) is formulated at a pH of between 3 and 6; and wherein said compound of formula (1) is formulated in said composition at a concentration of between 0.1% and 5% w/w.

26. The composition of claim 25, wherein said compound of formula (1) is formulated such that upon topical administration to a human at least 10% of said compound of formula (1) is retained within the skin 4 hours after administration.

27. The composition of claim 25, wherein said compound of formula (1) is formulated such that upon topical administration to a human the systemic exposure of said human to said compound of formula (1) is less than 20 ng/ml.

28. The composition of claim 25, wherein said compound of formula (1) is nalmefene methiodide or nalmefene hydrochloride.

29. A topical pharmaceutical composition comprising a salt of the compound of formula (1).

30. The topical pharmaceutical composition of claim 29, wherein R1 is alkyl or alkylcycloalkyl.

31. The topical pharmaceutical composition of claim 29, wherein the composition is aqueous and has a pH of between 3 and 7.

32. The topical pharmaceutical composition of claim 29, wherein X is halide.

33. The topical pharmaceutical composition of claim 29, wherein the compound of formula (1) is selected from nalmefene methiodide and nalmefene hydrochloride.

34. The topical pharmaceutical composition of claim 29, in the form of an ointment, lotion, or cream.

35. The topical pharmaceutical composition of claim 29, wherein said compound of formula (1) is more than 95% ionised.

36. The topical pharmaceutical composition of claim 29, wherein the concentration of the compound of formula (1) is between 0.1 to 5% w/w.

37. The topical pharmaceutical composition of claim 29, wherein said compound of formula (1) is formulated for once or twice daily administration.
